(19) United States
(12) Patent Application Publication
(10) Pub. No.: US 2009/0274641 A1
(43) Pub. Date: Nov. 5, 2009

(54) COSMETIC COMPOSITION COMPRISING ONE OR MORE VINYLFORMAMIDE/VINYLFORMAMINE COPOLYMERS AND ONE OR MORE THICKENING POLYMERS

(76) Inventors: Estelle Mathonneau, Paris (FR); Henri Samain, Bievres (FR); Katarina Benabdillah, Le Plessis-Bouchard (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/362,848

(22) Filed: Jan. 30, 2009

Related U.S. Application Data
(60) Provisional application No. 61/006,942, filed on Feb. 7, 2008.

(30) Foreign Application Priority Data
Jan. 31, 2008 (FR) 0850607

(51) Int. Cl. A61K 8/73; A61K 8/88; A61K 8/85; A61K 8/81; A61Q 5/06

(52) U.S. Cl. 424/70.13; 424/70.17; 424/70.16; 424/70.15

(57) ABSTRACT

A cosmetic composition. In a cosmetically acceptable medium:
one or more vinylformamide/vinylformamine copolymers, and
one or more thickening polymers different from the vinylformamide/vinylformamine copolymers.

Can be used for hair styling, and can make it possible to obtain a particularly lasting retention of the hair style and hair conditioning properties.

This application claims benefit of U.S. Provisional Application No. 61/006,942, filed Feb. 7, 2008, the contents of which are incorporated herein by reference. This application also claims benefit of priority under 35 U.S.C. § 119 to French Patent Application No. FR 0850607, filed Jan. 31, 2008, the contents of which are also incorporated herein by reference.

The present disclosure relates to a cosmetic composition comprising, in a cosmetically acceptable medium, one or more vinylformamide/vinylformamine copolymers and one or more thickening polymers.

The present disclosure also relates to a method of cosmetic treatment of the hair, for example, a method of fixing and/or shaping the hair using the abovementioned composition.

The present disclosure finally relates to a use of this composition for the cosmetic treatment of hair, such as for hairstyling which includes but is not limited to shaping and/or fixing the hair style.

Hair styling products are normally used to construct and structure the hair style and give it a lasting retention. They are customarily in the form of lotions, gels, mousses, creams, sprays and the like. The corresponding compositions generally comprise one or more film-forming polymers or “fixing polymers”, in a cosmetically acceptable medium. These polymers allow the formation of a film coating the hair, thus ensuring that the hair style is maintained.

However, the films of fixing polymer thus formed may have the disadvantage of being relatively brittle, which limits, over time, the retention of the hair style, and brings about the formation of unaesthetic residues on the hair.

Thus, conventional hair styling products may lead to a fixing of the hair style and to hair styling effects which gradually fade over time. In particular, when the product is applied in the morning, the hair styling effects gradually fade as the day progresses. On the next day, the hair styling effects are weak or even nonexistent.

To overcome this problem, it is known to incorporate into hair styling products polymers with a very high fixing power, and/or to increase the concentration of fixing polymer. However, the use of such extremely fixing products may cause a number of disadvantages. In particular, these products may lead to a dry and rough feel for the hair and are difficult to remove with shampoo.

A need therefore exists for hair compositions which make it possible to obtain a lasting fixing of the hair style, with hair styling effects which persist throughout the day or even for several days, while being easy to remove with shampoo and while providing a pleasant cosmetic feel, and in particular a smooth feel.

International Patent Application WO 96/03969 describes in a general way cosmetic compositions intended for fixing and/or conditioning the hair, which comprise a vinylformamide homopolymer or a copolymer of vinylformamide and one or more other vinyl monomers, in combination with at least one ingredient chosen from conditioning agents, emulsifying agents, surfactants, viscosity modifiers, gelling agents, opacifying agents, stabilizing agents, preservatives, sequestering agents, chelating agents, pearlescent agents, clarifying agents, perfumes, colorants, propellants, organic solvents and water.

Moreover, American U.S. Pat. No. 4,421,602 describes vinylformamide/vinylformamine copolymers, their preparation and their use in the paper industry to improve retention, flowability and flocculation.

It has now been discovered, surprisingly, that the combination of a vinylformamide/vinylformamine copolymer with a thickening polymer in a non-cleansing cosmetic composition (that is to say with a low content of anionic and non-ionic surfactants, or even free of such surfactants) made it possible to obtain a cosmetic hair composition providing improved hair styling properties. For example, such a combination makes it possible to obtain hair styling products providing a lasting fixing of the hair style, while being easy to remove during washing and providing a pleasant cosmetic feel to the hair after shampooing.

The present disclosure thus makes it possible to prepare hair styling products which provide very high levels of fixing, a very long retention of the hair style over time, and good resistance thereof to mechanical stresses. After shampooing, the hair feels particularly soft, and the hair is disentangled.

An embodiment of the present invention is therefore a cosmetic composition comprising, in a cosmetically acceptable medium:

one or more vinylformamide/vinylformamine copolymers comprising:

from 10 to less than 95 mol % of units of the following formula A:

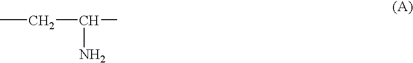

and from 90 to greater than 5 mol % of units of the following formula B:

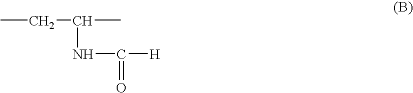

one or more thickening polymers different from the vinylformamide/vinylformamine copolymers.

For example, this composition may contain less than 5% by weight in total of anionic surfactants and of non-ionic surfactants.

The compositions make it possible, for example to perform hair styles lock by lock on hair, such as on short hair, which hair styles can withstand mechanical stresses particularly well and exhibit a supple or hard fixing according to the concentrations of the vinylformamide/vinylformamine copolymer and of thickening polymer.

Other subjects, characteristics, aspects and advantages of the invention will emerge even more clearly on reading the description and examples which follow.

According to at least one embodiment, the cosmetic composition comprises, in a cosmetically acceptable medium, one or more vinylformamide/vinylformamine copolymers, and one or more thickening polymers different from the vinylformamide/-vinylformamine copolymers.

The expression “cosmetically acceptable medium” is understood to mean a medium compatible with keratin materials, for example, the hair.

For example, the cosmetically acceptable medium comprises water and/or one or more cosmetically acceptable solvents chosen from C₁-C₄ lower alcohols, such as ethanol, isopropanol, tert-butanol and n-butanol; polyols such as propylene glycol; polyol ethers; C₅-C₁₀ alkanes; C₃₋₄ ketones such as acetone and methyl ethyl ketone; C₁-C₄ alkyl acetates such as methyl acetate, ethyl acetate and butyl acetate; dimethoxyethane, diethoxyethane; and mixtures thereof.

### Vinylformamide/Vinylformamine Copolymers

The vinylformamide/vinylformamine copolymer(s) which can be used in the compositions comprise, for example, from 10 to 60 mol % of unit of formula A such as from 20 to 40 mol %.

The vinylformamide/vinylformamine copolymer(s) can comprise, for example, from 30 to 90 mol % of unit of formula B such as from 60 to 80 mol %.

The copolymers may be obtained, for example, by partial hydrolysis of polyvinylformamide. This hydrolysis may be performed in acidic or basic medium.

The vinylformamide/vinylformamine copolymer(s) may optionally comprise one or more additional monomer units. In this case, the latter can represent less than 20 mol % of the copolymer.

According to at least one embodiment, the vinylformamide/vinylformamine copolymer(s) are constituted solely of units of formula A and of units of formula B.

The weight-average molecular mass of the said copolymer, measured by diffraction of light, may vary from 10 000 to 30 000 000 g/mol, such as from 40 000 to 1 000 000 and further such as from 100 000 to 500 000 g/mol.

The density of cationic charge of the said copolymer may vary from 2 meq/g to 20 meq/g, such as from 2.5 to 15 and further such as from 3.5 to 10 meq/g.

By way of example of vinylformamide/vinylformamine copolymers which can be used in the compositions, there may be mentioned, inter alia, the products marketed under the name LUPAMIN by the company BASF, such as for example, and without limitation, the products provided under the name LUPAMIN 9030, LUPAMIN 9010 and LUPAMIN 5095.

The vinylformamide/vinylformamine copolymer(s) are present, for example, in the compositions in amounts ranging from 0.1 to 25% by weight, such as from 0.5 to 20% by weight and further such as from 1 to 10% by weight, relative to the total weight of the composition.

### Thickening Polymers

The expression “thickening polymer” is understood to mean a polymer which, when introduced at 1% in a pure aqueous solution or an aqueous-alcoholic solution containing 30% of ethanol, and at pH=7, makes it possible to reach a viscosity of at least 100 cps, preferably at least 500 cps, at 25° C. and at a shear rate of 1s⁻¹. This viscosity may be measured with the aid of a cone/plate viscometer (Rheometer Haake R600 or the like).

For example, these polymers increase, by their presence, the viscosity of compositions into which they are introduced by at least 50 cps, such as 200 cps, at 25° C., and at a shear rate of 1 s⁻¹.

The thickening polymers may be ionic or non-ionic, associative or non-associative polymers.

The non-associative thickening polymers are thickening polymers which do not contain a C₁₀-C₃₀ fatty chain.

Among the non-associative thickening polymers present, there may be mentioned crosslinked homopolymers or copolymers of acrylic or methacrylic acid, crosslinked homopolymers of 2-acrylamido-2-methylpropanesulphonic acid and their crosslinked copolymers of acrylamide, homopolymers of ammonium acrylate or copolymers of ammonium acrylate and acrylamide, non-ionic guar gums, biopolysaccharide gums of microbial origin, gums derived from plant exudates, celluloses, in particular hydroxypropyl- or carboxymethylcelluloses; pectins and alginates, alone or as mixtures.

A first family of suitable non-associative thickening polymers is represented by the crosslinked homopolymers of acrylic acid.

Among the homopolymers of this type, there may be mentioned those crosslinked with an allyl ether of an alcohol of the sugar series, such as for example the products sold under the names CARBOPOLS 980, 981, 954, 2984 and 5984 by the company NOVEON or the products sold under the names SYNTHALEN M and SYNTHALEN K by the company 3 VSA.

The non-associative thickening polymers may also be crosslinked copolymers of (meth)acrylic acids such as the polymer sold under the name AQUA SF1 by the company NOVEON.

The non-associative thickening polymers may be chosen from the crosslinked homopolymers of 2-acrylamido-2-methylpropanesulphonic acid and their crosslinked copolymers of acrylamide.

As regards these homopolymers and copolymers, which may be partially or completely neutralized, there may be mentioned the polymers comprising from 90 to 99.9% by weight, relative to the total weight of the polymer, of units of the following formula (j):

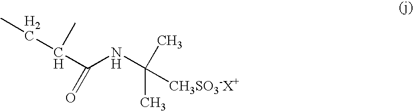

in which X⁺ denotes a cation or a mixture of cations, or a proton.

For example, the cations are chosen from alkali metals (such as sodium, potassium), ammonium ions which are unsubstituted or substituted with one to three alkyl radicals, which may be identical or different, comprising from 1 to 6 carbon atoms, optionally carrying at least one hydroxyl radical, the cations being derived from N-methylglucamine, basic amino acids such as arginine and lysine. As a further example, the cation is an ammonium or sodium ion.

Moreover, the polymer comprises from 0.01 to 10% by weight, relative to the total weight of the polymer, of crosslinking units derived from at least one monomer having at least two ethylenic unsaturations (carbon-carbon double bond).

The crosslinking monomers having at least two ethylenic unsaturations are chosen, for example, from diallyl ether, triallyl cyanurate, diallyl maleate, allyl (meth)acrylate, dipropylene glycol diallyl ether, polyglycol diallyl ethers, triethylene glycol divinyl ether, hydroquinone diallyl ether, tetrallyl-oxethanoyl, tetra- or diethylene glycol di(meth)acrylate, triallylamine, tetraallylethylenediamine, trimethylolpropane diallyl ether, trimethylolpropane triacrylate, methylene-bis(meth)acrylamide or divinylbenzene, allyl ethers of alcohols of the sugar series, or other allyl- or vinyl ethers of polyfunctional alcohols, and allyl esters of phosphoric and/or vinylphosphonic acid derivatives, or mixtures of these compounds.

For more details on the subject of these polymers, reference may be made to the document EP 815828.

Among the partially or completely neutralized crosslinked copolymers of 2-acrylamido-2-methylpropanesulphonic acid and acrylamide, there may be mentioned, for example, the product described in Example 1 of the document EP 503 853 and reference may be made to this document as regards these polymers.

The composition may likewise comprise, as non-associative thickening polymers, homopolymers of ammonium acrylate or copolymers of ammonium acrylate and acrylamide.

As examples of homopolymers of ammonium acrylate, there may be mentioned the product sold under the name MICROSAP PAS 5193 by the company HOECHST. Among the copolymers of ammonium acrylate and acrylamide, there may be mentioned the product sold under the name BOZEPOL C NOUVEAU or the product PAS 5193 sold by the company HOECHST. Reference may be made, for example, to the documents FR 2 416 723, U.S. Pat. No. 2,798,053 and U.S. Pat. No. 2,923,692 as regards the description and preparation of such compounds.

The composition may also comprise homopolymers of dimethylaminoethyl methacrylate quaternized with methyl chloride or the copolymers of dimethylaminoethyl methacrylate quaternized with methyl chloride and of acrylamide.

Among the homopolymers of this type, there may be mentioned the products sold under the names SALCARE SC95 and SALCARE SC96 by the company CIBA. Among the copolymers of this family, there may be mentioned the product SALCARE SC92 sold by CIBA or the product PAS 5194 sold by HOECHST. These polymers are described and prepared, for example, in the document EP 395282 to which reference may be made.

As non-associative thickening polymers, there may be mentioned non-ionic guar gums, such as for example the unmodified non-ionic guar gums sold under the name VIDOGUM GH 175 by the company UNIPECTINE and under the name JAGUAR C by the company MEYHALL.

The non-ionic guar gums can be modified with C₁-C₆ hydroxyalkyl groups. Among the hydroxyalkyl groups, there may be mentioned, by way of example, the groups hydroxymethyl, hydroxyethyl, hydroxypropyl and hydroxybutyl.

These guar gums are well known in the state of the art and may, for example, be prepared by reacting corresponding alkene oxides, such as for example propylene oxides, with guar gum, so as to obtain a guar gum modified with hydroxypropyl groups.

The rate of hydroxyalkylation, which corresponds to the number of alkylene oxide molecules consumed per number of free hydroxyl functional groups present on the guar gum, can vary from 0.4 to 1.2.

Such non-ionic guar gums optionally modified with hydroxyalkyl groups are for example sold under the trade names JAGUAR HP8, JAGUAR HP60 and JAGUAR HP120, JAGUAR DC 293 and JAGUAR HP 105 by the company MEYHALL or under the name GALACTASOL 4H4FD2 by the company AQUALON.

As suitable non-associative thickening polymers, there may also be mentioned the biopolysaccharide gums of microbial origin such as scleroglucan or xanthan gums.

Also suitable are the gums derived from plant exudates, such as gum arabic, Ghatti gums, Karaya and Tragacanth gums; celluloses, such as hydroxypropyl- or carboxymethylcelluloses; pectins and alginates.

These polymers are well known to a person skilled in the art and are described in particular in the manual by Robert L. DAVIDSON entitled “Handbook of Water soluble gums and resins” published by McGraw Hill Book Company (1980).

Representative thickening agents, include the thickening systems based on associative polymers well known to a person skilled in the art and can be of non-ionic, anionic, cationic or amphoteric nature.

Representative associative polymers are hydrophilic polymers which are capable, in an aqueous medium, of reversibly combining with each other or with other molecules.

Their chemical structure can comprise at least one hydrophilic region and at least one hydrophobic region.

The expression hydrophobic group is understood to mean a radical or polymer having a saturated or unsaturated, linear or branched hydrocarbon chain, comprising, for example, at least 10 carbon atoms, such as from 10 to 30 carbon atoms, further such as from 12 to 30 carbon atoms and even further such as from 18 to 30 carbon atoms.

For example, the hydrocarbon group is derived from a monofunctional compound. By way of example, the hydrophobic group may be derived from a fatty alcohol such as stearyl alcohol, dodecyl alcohol or decyl alcohol. It may also denote a hydrocarbon polymer such as for example polybutadiene.

Among the associative polymers of the anionic type, there may be mentioned:

(I) those containing at least one hydrophilic unit, and at least one fatty chain allyl ether, such as those whose hydrophilic unit consists of an ethylenic unsaturated anionic monomer, for example, of a vinylcarboxylic acid and as a further example, of an acrylic acid or a methacrylic acid or mixtures thereof, and whose fatty chain allyl ether unit corresponds to the monomer of the following formula (I):

CH₂═CR′CH₂OBₙR (I)

in which R′ denotes H or CH₃, B denotes the ethyleneoxy radical, n is zero or denotes an integer ranging from 1 to 100, R denotes a hydrocarbon radical chosen from alkyl, arylalkyl, aryl, alkylaryl and cycloalkyl radicals comprising, for example from 8 to 30 carbon atoms, such as from 10 to 24, and further such as from 12 to 18 carbon atoms. A unit of formula (I) can be a unit in which R′ denotes H, n is equal to 10, and R denotes a stearyl (C₁₈) radical.

Anionic associative polymers of this type are described and prepared, according to an emulsion polymerization method, in Patent EP-0 216 479.

Representative anionic associative polymers can be polymers formed from 20 to 60% by weight of acrylic acid and/or methacrylic acid, from 5 to 60% by weight of lower alkyl (meth)acrylates, from 2 to 50% by weight of fatty chain allyl ether of formula (I), and from 0 to 1% by weight of a crosslinking agent which is a well known copolymerizable polyethylenic unsaturated monomer, such as diallyl phthalate, allyl (meth)acrylate, divinylbenzene, (poly)ethylene glycol dimethacrylate and methylene-bis-acrylamide.

Further representative anionic associative polymers can be the crosslinked terpolymers of methacrylic acid, ethyl acrylate and polyethylene glycol (10 EO) ether of stearyl alcohol (Steareth 10), for example, those sold by the company CIBA under the names SALCARE SC80® and SALCARE SC90® which are aqueous emulsions containing 30% of a crosslinked terpolymer of methacrylic acid, ethyl acrylate and steareth-10-allyl ether (40/50/10).

(II) those containing at least one hydrophilic unit of the olefinic unsaturated carboxylic acid type, and at least one hydrophobic unit of the (C₁₀-C₃₀) alkyl ester of unsaturated carboxylic acid type.

For example, these polymers are chosen from those whose hydrophilic unit of the olefinic unsaturated carboxylic acid type corresponds to the monomer of the following formula (II):

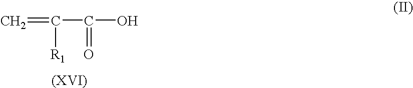

in which R₁ denotes H or CH₃ or C₂H₅, that is to say acrylic acid, methacrylic acid or ethacrylic acid units, and whose hydrophobic unit of the (C₁₀-C₃₀) alkyl ester of unsaturated carboxylic acid type corresponds to the monomer of the following formula (III)

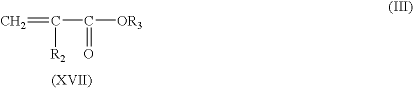

in which R2 denotes H or CH3 or C2H5 (that is to say acrylate, methacrylate or ethacrylate units) and preferably H (acrylate units) or CH₃ (methacrylate units), R₃ denoting a C₁₀-C₃₀, and preferably C₁₂-C₂₂, alkyl radical.

(C₁₀-C₃₀) alkyl esters of unsaturated carboxylic acids comprise, for example, lauryl acrylate, stearyl acrylate, decyl acrylate, isodecyl acrylate, dodecyl acrylate and the corresponding methacrylates, lauryl methacrylate, stearyl methacrylate, decyl methacrylate, isodecyl methacrylate and dodecyl methacrylate.

Anionic polymers of this type are for example described and prepared according to U.S. Pat. Nos. 3,915,921 and 4,509,949.

This type of anionic associative polymers are exemplified as the polymers formed from a mixture of monomers comprising:

essentially acrylic acid,

an ester of formula (III) described above and in which R₂ denotes H or CH₃, R₃ denoting an alkyl radical having from 12 to 22 carbon atoms, and

(iii) a crosslinking agent, which is a well known copolymerizable polyethylenic unsaturated monomer, such as diallyl phthalate, allyl (meth)acrylate, divinylbenzene, (poly)ethylene glycol dimethacrylate and methylene-bis-acrylamide.

This type of anionic associative polymers are also exemplified as those constituted of 95 to 60% by weight of acrylic acid (hydrophilic unit), 4 to 40% by weight of C₁₀-C₃₀ alkyl acrylate (hydrophobic unit), and 0 to 6% by weight of crosslinking polymerizable monomer, or those constituted of 98 to 96% by weight of acrylic acid (hydrophilic unit), 1 to 4% by weight of C₁₀-C₃₀ alkyl acrylate (hydrophobic unit), and 0.1 to 0.6% by weight of crosslinking polymerizable monomer such as those described above.

The said polymers above include but are not limited to the products sold by the company GOODRICH under the trade names PEMULEN TR1®, PEMULEN TR2®, CARBOPOL 1382®, such as PEMULEN TR1®, and the product sold by the company S.E.P.P.I.C. under the name COATEX SX®.

Mention may also be made of the polymers which, in addition to the monomers of formula (II) and of formula (III), contain one or more other monomers. This additional monomer may be a vinyllactam such as vinylpyrrolidone.

As an example of a polymer, there may be mentioned the acrylic acid/lauryl methacrylate/vinylpyrrolidone terpolymer marketed under the name Acrylidone LM by the company ISP.

(III) maleic anhydride/C₃₀-C₃₈ α-olefin/alkyl maleate terpolymers such as the product (maleic anhydride/C₃₀-C₃₈ α-olefin/isopropyl maleate copolymer) sold under the name PERFORMA V 1608® by the company NEWPHASE TECHNOLOGIES.

(IV) the acrylic terpolymers comprising:

(a) about 20% to 70% by weight of an α,β-monoethylenically unsaturated carboxylic acid,

(b) about 20 to 80% by weight of a non-surfactant α,β-monoethylenically unsaturated monomer different from (a),

(c) about 0.5 to 60% by weight of a non-ionic monourethane which is the product of the reaction of a monohydric surfactant with a monoethylenically unsaturated monoisocyanate,

such as those described in Patent Application EP-A-0173109, for example, that described in Example 3, namely a methacrylic acid/methyl acrylate/ethoxylated behenyl alcohol dimethyl meta-isopropenyl benzyl isocyanate (40EO) terpolymer as a 25% aqueous dispersion.

(V) copolymers containing, among their monomers, an α,β-monoethylenically unsaturated carboxylic acid and an ester of an α,β-monoethylenically unsaturated carboxylic acid and an oxyalkylenated fatty alcohol.

As a further example, these compounds also comprise, as monomer, an ester of an α,β-monoethylenically unsaturated carboxylic acid and a C₁-C₄ alcohol.

By way of example of this type of compound, there may be mentioned ACULYN 22® sold by the company ROHM and HAAS, which is a methacrylic acid/ethyl acrylate/oxyalkylenated stearyl methacrylate terpolymer.

Among the cationic type associative polymers, there may be mentioned:

(I) cationic associative polyurethanes the family of which has been described by the applicant in French Patent Application No. 0009609; it may be represented by the following general formula (IV):

R—X—(P)n-[L-(Y)m]r-L′—(P′)p-X′—R′ (IV)

in which:

R and R′, which are identical or different, represent a hydrophobic group or a hydrogen atom;

X and X′, which are identical or different, represent a group containing an amine functional group bearing or not bearing a hydrophobic group, or the group L″;

L, L′ and L″, which are identical or different, represent a group derived from a diisocyanate;

P and P′, which are identical or different, represent a group containing an amine functional group bearing or not bearing a hydrophobic group;

Y represents a hydrophilic group;

r is an integer ranging from 1 to 100, such as from 1 to 50 and further such as from 1 to, 25,

n, m, and p are each, independently of the others, ranging from 0 to 1000;

the molecule containing at least one protonated or quaternized amine functional group and at least one hydrophobic group.

In at least one embodiment of these polyurethanes, the sole hydrophobic groups are the groups R and R′ at the chain ends.

A representative family of cationic associative polyurethanes is that corresponding to the formula (IV) described above and in which:

R and R′ both independently represent a hydrophobic group,

X, X′ each represent a group L″,

n and p are between 1 and 1000, and

L, L′, L″, P, P′, Y and m have the meaning indicated above.

Another representative family of cationic associative polyurethanes is that corresponding to the above formula (IV) in which:

R and R′ both independently represent a hydrophobic group, X, X′ each represent a group L″, n and p are 0, and L, L′, L″, Y and m have the meaning indicated above.

The fact that n and p are 0 means that these polymers do not contain units derived from an amine functional group-containing monomer incorporated into the polymer during polycondensation. The protonated amine functional groups of these polyurethanes result from the hydrolysis of the isocyanate functional groups, in excess, at the chain end, followed by alkylation of the primary amine functional groups formed by hydrophobic group-containing alkylating agents, that is to say compounds of the RQ or R′Q type, in which R and R′ are as defined above and Q denotes a leaving group such as a halide, a sulphate and the like.

Yet another representative family of cationic associative polyurethanes is that corresponding to the above formula (Ia) in which:

R and R′ both independently represent a hydrophobic group,

X and X′ both independently represent a group containing a quaternary amine,

n and p are zero, and

L, L′, Y and m have the meaning indicated above.

The number-average molecular mass of the cationic associative polyurethanes is, for example, ranging from 400 to 500 000, such as from 1000 to 400 000 and further such as from 1000 to 300 000.

The expression hydrophobic group is understood to mean a radical or polymer containing a saturated or unsaturated, linear or branched hydrocarbon chain, which may contain one or more heteroatoms such as P, O, N, S, or a radical having a perfluorinated or silicone chain. When it denotes a hydrocarbon radical, the hydrophobic group contains at least 10 carbon atoms, such as from 10 to 30 carbon atoms, further such as from 12 to 30 carbon atoms and even further such as from 18 to 30 carbon atoms.

For example, the hydrocarbon group is derived from a monofunctional compound.

By way of example, the hydrophobic group may be derived from a fatty alcohol such as stearyl alcohol, dodecyl alcohol or decyl alcohol. It may also denote a hydrocarbon polymer such as for example polybutadiene.

When X and/or X′ denote a group containing a tertiary or quaternary amine, X and/or X′ may represent one of the following formulae:

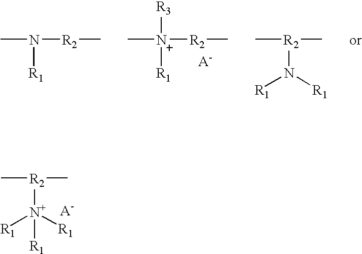

for x

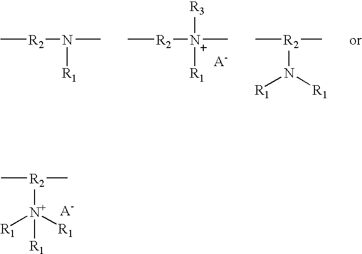

for X′

in which:

R₂ represents a linear or branched alkylene radical having from 1 to 20 carbon atoms, containing or not containing a saturated or unsaturated ring, or an arylene radical, it being possible for one or more of the carbon atoms to be replaced by a heteroatom chosen from N, S, O, P;

R₁ and R₃, which are identical or different, denote a linear or branched C₁-C₃₀ alkyl or alkenyl radical, an aryl radical, it being possible for at least one of the carbon atoms to be replaced by a heteroatom chosen from N, S, O, P;

A⁻ is a physiologically acceptable counterion.

The groups L, L′ and L″ represent a group of formula:

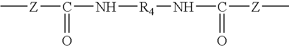

in which:

Z represents —O—, —S— or —NH—; and

R₄ represents a linear or branched alkylene radical having from 1 to 20 carbon atoms, containing or not containing a saturated or unsaturated ring, an arylene radical, it being possible for one or more of the carbon atoms to be replaced by a heteroatom chosen from N, S, O and P.

The groups P and P′, comprising an amine functional group, may represent at least one of the following formulae:

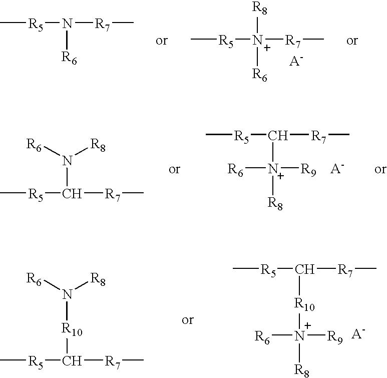

in which:

R₅ and R₇ have the same meanings as R₂ defined above;

R₆, R₈ and R₉ have the same meanings as R₁ and R₃ defined above;

R₁₀ represents an optionally unsaturated linear or branched alkylene group which may contain one or more heteroatoms chosen from N, O, S and P,

and A⁻ is a physiologically acceptable counterion.

As regards the meaning of Y, the expression hydrophilic group is understood to mean a polymeric or non-polymeric water-soluble group.

By way of example there may be mentioned, when polymers are not involved, ethylene glycol, diethylene glycol and propylene glycol.

When, in accordance with at least one embodiment, a hydrophilic polymer is involved, there may be mentioned, by way of example, polyethers, sulphonated polyesters, sulphonated polyamides, or a mixture of these polymers. For example, the hydrophilic compound can be a polyether such as a poly(ethylene oxide) or poly(propylene oxide).

The cationic associative polyurethanes of formula (IV) are, for example, formed from diisocyanates and from various compounds possessing functional groups having a labile hydrogen. The functional groups having a labile hydrogen may be alcohol, primary or secondary amine or thiol functional groups which give, after reaction with the diisocyanate functional groups, polyurethanes, polyureas and polythioureas, respectively. The term “polyurethanes” covers these three types of polymer, namely the polyurethanes proper, the polyureas and the polythioureas and copolymers thereof.

A first type of compounds entering into the preparation of the polyurethane of formula (IV) can be a compound containing at least one unit having an amine functional group. This compound may be multifunctional, for example, the compound may be difunctional, that is to say that according to at least one embodiment, this compound contains two labile hydrogen atoms carried for example by a hydroxyl, primary amine, secondary amine or thiol functional group. It is also possible to use a mixture of multifunctional and difunctional compounds in which the percentage of multifunctional compounds is low.

As indicated above, this compound may contain more than one unit having an amine functional group. It is then a polymer bearing a repeat of the unit having an amine functional group.

This type of compound may be represented by one of the following formulae:

HZ-(P)n-ZH,

or

HZ-(P′)p-ZH

in which Z, P, P′, n and p are as defined above.

By way of example of a compound having an amine functional group, there may be mentioned N-methyldiethanolamine, N-tert-butyldiethanolamine, N-sulphoethyldiethanolamine.

The second compound entering into the preparation of the polyurethane of formula (IV) can be a diisocyanate corresponding to the formula:

O═C═N—R₄—N═C═O

in which R₄ is defined above.

By way of example, there may be mentioned methylenediphenyl diisocyanate, methylenecyclohexane diisocyanate, isophorone diisocyanate, toluene diisocyanate, naphthalene diisocyanate, butane diisocyanate, hexane diisocyanate.

A third compound entering into the preparation of the polyurethane of formula (IV) can be a hydrophobic compound intended to form hydrophobic end groups of the polymer of formula (IV).

This compound may be constituted of a hydrophobic group and a functional group having a labile hydrogen, for example a hydroxyl, primary or secondary amine or thiol functional group.

By way of example, this compound may be a fatty alcohol, such as stearyl alcohol, dodecyl alcohol or decyl alcohol. When this compound contains a polymeric chain, it may be for example alpha-hydroxyl hydrogenated polybutadiene.

The hydrophobic group of the polyurethane of formula (IV) may also result from the quaternization reaction of the tertiary amine of the compound containing at least one tertiary amine unit. Thus, the hydrophobic group can be introduced by the quaternizing agent. This quaternizing agent can be a compound of the RQ or R′Q type, in which R and R′ are as defined above and Q denotes a leaving group such as a halide, a sulphate or the like.

The cationic associative polyurethane may additionally comprise a hydrophilic block. This block can be provided by a fourth type of compound entering into the preparation of the polymer. This compound may be multifunctional. It may be, for example, difunctional. It is also possible to have a mixture where the percentage of multifunctional compound is low.

The functional groups having a labile hydrogen can be alcohol, primary or secondary amine or thiol functional groups. This compound may be a polymer terminated at the chain ends by one of these functional groups having a labile hydrogen.

By way of example, there may be mentioned, when polymers are not involved, ethylene glycol, diethylene glycol and propylene glycol.

In the case of a hydrophilic polymer, there may be mentioned, by way of example, polyethers, sulphonated polyesters, sulphonated polyamides, or a mixture of these polymers. For example, the hydrophilic compound may be a polyether such as a poly(ethylene oxide) or poly(propylene oxide).

The hydrophilic group noted Y in the formula (IV) may be optional. Indeed, the units having a quaternary or protonated amine functional group may be sufficient to provide the solubility or water-dispersibility necessary for this type of polymer in an aqueous solution.

Although the presence of a hydrophilic group Y may be optional, cationic associative polyurethanes may contain such a group.

(II) quaternized cellulose derivatives and polyacrylates having non-cyclic amino side groups.

The quaternized cellulose derivatives may be,

quaternized celluloses modified by groups containing at least one fatty chain, such as alkyl, arylalkyl or alkylaryl groups containing at least 8 carbon atoms, or mixtures thereof,

quaternized hydroxyethylcelluloses modified by groups containing at least one fatty chain, such as alkyl, arylalkyl or alkylaryl groups containing at least 8 carbon atoms, or mixtures thereof.

The alkyl radicals carried by the above quaternized celluloses or hydroxyethylcelluloses, for example, contain from 8 to 30 carbon atoms. The aryl radicals, for example, denote phenyl, benzyl, naphthyl or anthryl groups.

It is possible to mention, as examples of quaternized alkylhydroxyethylcelluloses having C₈-C₃₀ fatty chains, the products QUATRISOFT LM 200®, QUATRISOFT LM-X 529-18-A®, QUATRISOFT LM-X 529-18B® (C₁₂ alkyl) and QUATRISOFT LM-X 529-8® (C₁₈ alkyl) marketed by the company AMERCHOL and the products CRODACEL QM®, CRODACEL QL® (C₁₂ alkyl) and CRODACEL QS® (C₁₈ alkyl) marketed by the company CRODA.

(III) the cationic polymer(s) obtained by polymerization of a mixture of monomers comprising one or more vinyl monomers substituted with one or more amino groups, one or more hydrophobic non-ionic vinyl monomers, and one or more associative vinyl monomers.

For example, among these cationic polymers, there may be mentioned the compound marketed by the company NOVEON under the name AQUA CC and which corresponds to the INCI name POLYACRYLATE-1 CROSSPOLYMER.

POLYACRYLATE-1 CROSSPOLYMER is the product of the polymerization of a mixture of monomers comprising:

a di(C₁-C₄ alkyl)amino(C₁-C₆ alkyl)methacrylate,

one or more (meth)acrylic acid and C₁-C₃₀ alkyl esters,

a polyethoxylated C₁₀-C₃₀ alkyl methacrylate (20-25 moles of ethylene oxide unit),

an allyl ether of 30/5 polyethylene glycol/polypropylene glycol,

a hydroxy(C₂-C₆ alkyl)methacrylate, and

an ethylene glycol dimethacrylate.

The amphoteric associative polymers are, for example, chosen from those containing at least one non-cyclic cationic unit. Exemplary mention may also be made of those prepared from or comprising 1 to 20 mol % of monomer containing a fatty chain, such as from 1.5 to 15 mol % and further such as from 1.5 to 6 mol %, relative to the total number of moles of monomers.

The representative amphoteric associative polymers comprise, or are prepared by copolymerizing at least one monomer of formula (V) or (VI):

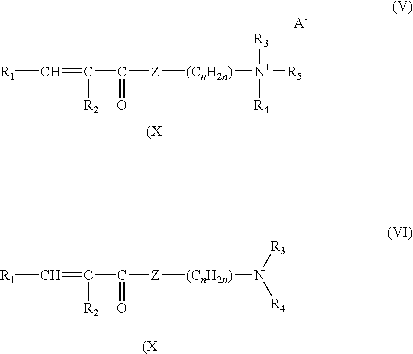

in which R₁ and R₂, which are identical or different, represent a hydrogen atom or a methyl radical, R₃, R₄ and R₅, which are identical or different, represent a linear or branched alkyl radical having from 1 to 30 carbon atoms,

Z represents an NH group or an oxygen atom,

n is an integer from 2 to 5,

A⁻ is an anion derived from an organic or inorganic acid, such as a methosulphate anion or a halide such as chloride or bromide;

2) at least one monomer of formula (VIII)

R₆—CH═CR₇—COOH (X (VII)

in which R₆ and R₇, which are identical or different, represent a hydrogen atom or a methyl radical;

and

3) at least one monomer of formula (VIII):

R₆—CH═CR₇—COXR₈ (VIII)

in which R₆ and R₇, which are identical or different, represent a hydrogen atom or a methyl radical, X denotes an oxygen or nitrogen atom and R₈ denotes a linear or branched alkyl radical having from 1 to 30 carbon atoms;

at least one of the monomers of formula (V), (VI) or (VII) containing at least one fatty chain.

The monomers of formula (V) and (VI) can be, for example, chosen from

dimethylaminoethyl methacrylate, dimethylaminoethyl acrylate,

diethylaminoethyl methacrylate, diethylaminoethyl acrylate,

dimethylaminopropylmethacrylate, dimethylaminopropyl acrylate,

dimethylaminopropylmethacrylamide, dimethylaminopropylacrylamide,

these monomers being optionally quaternized, for example, with a C₁-C₄ alkyl halide or a C₁-C₄ dialkyl sulphate.

As a further example, the monomer of formula (V) may be chosen from acrylamidopropyltrimethylammonium chloride and methacrylamido-propyltrimethylammonium chloride.

The monomers of formula (VII) may be chosen from acrylic acid, methacrylic acid, crotonic acid and 2-methylcrotonic acid. For example, the monomer of formula (VII) is acrylic acid.

The monomers of formula (VIII) may be chosen from C₁₂-C₂₂, such as C₁₆-C₁₈, alkyl acrylates or methacrylates.

The monomers constituting the fatty chain-containing amphoteric polymers of the invention may be already neutralized and/or quaternized.

The ratio of the number of cationic charges/anionic charges may be, for example, equal to about 1.

The amphoteric associative polymers, for example, comprise from 1 to 10 mol % of the monomer containing a fatty chain (monomer of formula (V), (VI) or (VIII)), such as from 1.5 to 6 mol %.

The weight-average molecular weights of the amphoteric associative polymers may vary from 500 to 50 000 000, such as from 10 000 to 5 000 000.

The amphoteric associative polymers may also contain other monomers such as non-ionic monomers, such as C₁-C₄ alkyl acrylates or methacrylates.

Amphoteric associative polymers are for example described and prepared in Patent Application WO9844012.

Representative amphoteric associative polymers can be the acrylic acid/(meth)acrylamidopropyltrimethylammonium chloride/stearyl methacrylate terpolymers.

The non-ionic type associative polymers which can be used may be chosen from:

(1) celluloses modified with groups containing at least one fatty chain;

there may be mentioned by way of example:

hydroxyethylcelluloses modified with groups containing at least one fatty chain such as alkyl, arylalkyl or alkylaryl groups, or mixtures thereof, and in which the alkyl groups are, for example, C₈-C₂₂, such as the product NATROSOL PLUS GRADE 330 CS® (C₁₆ alkyls) sold by the company AQUALON, or the product BERMOCOLL EHM 100® sold by the company BEROL NOBEL,

those modified with alkyl phenol polyalkylene glycol ether groups, such as the product AMERCELL POLYMER HM-1500® (nonylphenol polyethylene glycol (15) ether) sold by the company AMERCHOL.

(2) hydroxypropyl guars modified with groups containing at least one fatty chain, such as the product ESAFLOR HM 220 (C₂₂ alkyl chain) sold by the company LAMBERTI, the products RE210-18® (C₁₄ alkyl chain) and RE205-1® (C₂₀ alkyl chain) sold by the company RHONE POULENC.

(3) copolymers of vinylpyrrolidone and hydrophobic monomers having a fatty chain, of which there may be mentioned by way of example:

the products ANTARON V216® or GANEX V216® (vinylpyrrolidone/hexadecene copolymer) sold by the company I.S.P.

the products ANTARON V220® or GANEX V220® (vinylpyrrolidone/eicosene copolymer) sold by the company I.S.P.

(4) copolymers of C₁-C₆ alkyl methacrylates or acrylates and amphiphilic monomers containing at least one fatty chain, such as for example the oxyethylenated stearyl acrylate/methyl acrylate copolymer sold by the company GOLDSCHMIDT under the name ANTIL 208®.

(5) copolymers of hydrophilic methacrylates or acrylates and hydrophobic monomers containing at least one fatty chain, such as for example the polyethylene glycol methacrylate/lauryl methacrylate copolymer.

(6) polyether-polyurethanes containing in their chain both hydrophilic blocks, for example of a polyoxyethylenated nature and hydrophobic blocks which may be aliphatic linkages alone and/or cycloaliphatic and/or aromatic linkages.

(7) polymers having an aminoplast ether backbone possessing at least one fatty chain, such as the compounds PURE THIX® provided by the company SUD-CHEMIE.

For example, the polyether-polyurethanes contain at least two lipophilic hydrocarbon chains having from 6 to 30 carbon atoms, separated by a hydrophilic block, it being possible for the hydrocarbon chains to be pendant chains or chains at the end of the hydrophilic block. As a further example, it is possible for one or more pendant chains to be provided. In addition, the polymer may contain a hydrocarbon chain at one end or at both ends of the hydrophilic block.

The polyether-polyurethanes may be polyblocks, for example, in triblock form. The hydrophobic blocks may be at each end of the chain (for example: triblock copolymer with a hydrophilic central block) or distributed both at the ends and in the chain (polyblock copolymer for example). These same polymers may also be graft or star-shaped polymers.

The non-ionic polyether-polyurethanes having a fatty chain may be triblock copolymers whose hydrophilic block is a polyoxyethylenated chain containing from 50 to 1000 oxyethylenated groups. The non-ionic polyether-polyurethanes may contain a urethane bond between the hydrophilic blocks, hence the origin of the name.

Exemplary non-ionic polyether-polyurethanes having a fatty chain can be those whose hydrophilic blocks are linked to the lipophilic blocks by other chemical bonds.

By way of example of non-ionic polyether-polyurethanes having a fatty chain which can be used, it is also possible to use Rheolate 205® having a urea functional group sold by the company RHEOX or alternatively Rheolates® 208, 204 or 212, and Acrysol RM 184®.

Exemplary mention may also be made of the product ELFACOS T210® having a C₁₂₋₁₄ alkyl chain and the product ELFACOS T212® having a C₁₈ alkyl chain from AKZO.

The product DW 1206B® from ROHM & HAAS having a C₂₀ alkyl chain and a urethane bond, provided at 20% dry matter content in water, may also be used.

It is also possible to use solutions or dispersions of these polymers, such as in water or in an aqueous-alcoholic medium. By way of example of such polymers, there may be mentioned Rheolate® 255, Rheolate® 278 and Rheolate® 244 sold by the company RHEOX. It is also possible to use the products DW 1206F and DW 1206J provided by the company ROHM & HAAS.

The polyether-polyurethanes which can used can be for example, those described in the article by G. Formum, J. Bakke and F k. Hansen—Colloid Polym. Sci 271, 380.389 (1993).

Representative polyether-polyurethane may be obtained by polycondensation of at least three compounds comprising (i) at least one polyethylene glycol comprising from 150 to 180 moles of ethylene oxide, (ii) stearyl alcohol or decyl alcohol and (iii) at least one dilsocyanate.

Such polyether-polyurethanes are sold, for example, by the company ROHM & HAAS under the names Aculyn 46® and Aculyn 44® [ACULYN 46® is a polycondensate of polyethylene glycol containing 150 or 180 moles of ethylene oxide, stearyl alcohol and methylene-bis(4-cyclohexyl isocyanate) (SMDI), at 15% by weight in a matrix of maltodextrin (4%) and water (81%); ACULYN 44® is a polycondensate of polyethylene glycol containing 150 or 180 moles of ethylene oxide, decyl alcohol and methylene-bis(4-cyclohexyl isocyanate) (SMDI), at 35% by weight in a mixture of propylene glycol (39%) and water (26%)].

The thickening polymers may be chosen from non-ionic guar gums and associative polymers.

Among the associative polymers, mention may be made of the compounds of the family (II), such as the acrylic acid/lauryl methacrylate/vinylpyrrolidone terpolymers.

The thickening polymer(s) may be present in the composition in an amount ranging from 0.01 to 20% by weight, such as from 0.5 to 5% by weight relative to the total weight of the composition, and further such as from 1 to 4% by weight relative to the total weight of the composition.

For example, the weight ratio of the total quantity of vinylformamide/vinylformamine copolymer(s), on the one hand, to the total quantity of thickening polymers, on the other hand, may range from 0.1 to 20, such as from 1 to 15, and further such as from 1 to 8.

The compositions may also contain one or more fatty substances.

The expression fatty substances is understood to mean an organic compound which, at room temperature (25° C.) and at atmospheric pressure, is insoluble in water (that is to say has a solubility in water of less than 1% by weight and preferably less than 0.5% by weight), and is soluble in at least one organic solvent (for example ethanol, chloroform or benzene) at least 1% by weight.

The non-silicone fatty substances which can be used in the compositions are, for example, all the natural or synthetic, organic or inorganic, non-silicone oils, waxes or resins corresponding to this definition.

An oilcan be a lipophilic compound which is liquid at room temperature and exhibits a reversible solid/liquid change of state.

As oils which can be used in the composition, there may be mentioned for example:

hydrocarbon oils of animal origin, such as perhydrosqualene;

hydrocarbon oils of plant origin, such as liquid triglycerides of fatty acids containing from 4 to 10 carbon atoms such as triglycerides of heptanoic or octanoic acids or alternatively, for example, sunflower, maize, soybean, gourd, grapeseed, sesame, hazelnut, apricot, macadamia, arara, sunflower, castor and avocado oils, triglycerides of caprylic/capric acids such as, for example, those sold by the company Stearineries Dubois or those sold under the names Miglyol 810, 812 and 818 by the company Dynamit Nobel, jojoba oil, shea butter oil;

synthetic esters and ethers, for example, of fatty acids, such as the oils of formulae R⁶COOR⁷ and R⁶OR⁷ in which R⁶ represents a saturated or unsaturated hydrocarbon chain (for example, the residue of a fatty acid) containing from 8 to 29 carbon atoms, and R⁷ represents a branched or unbranched hydrocarbon chain containing from 3 to 30 carbon atoms; there may be mentioned for example Purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, heptanoates, octanoates and decanoates of fatty alcohols; polyol esters, such as propylene glycol dioctanoate, neopentyl glycol diheptanoate and diethylene glycol diisononanoate; and pentaerythritol esters such as pentaerythrityl tetraisostearate;

linear or branched hydrocarbons of mineral or synthetic origin, such as volatile or non-volatile paraffin oils and their derivatives, petroleum jelly, liquid petroleum jelly, polydecenes, hydrogenated polyisobutene such as parleam oil;

fluid fatty alcohols having from 8 to 26 carbon atoms, such as for example octyldodecanol, 2-butyloctanol, oleyl alcohol, linoleyl alcohol or linolenyl alcohol;

partially hydrocarbon-based fluorinated oils such as those described in the document JP-A-2 295912. As fluorinated oils, there may also be mentioned perfluoromethylcyclopentane and perfluoro-1,3-dimethylcyclohexane, for example sold under the names “FLUTEC PC1®” and “FLUTEC PC3®” by the company BNFL Fluorochemicals; perfluoro-1,2-dimethylcyclobutane; perfluoralkanes such as dodecafluoropentane and tetradecafluorohexane, for example sold under the names “PF 5050®” and “PF 5060®” by the company 3M, or alternatively bromoperfluorooctyl for example sold under the name “FORALKYL®” by the company Atochem; nanofluoromethoxybutane for example sold under the name “MSX 4518®” by the company 3M and nanofluoroethoxyisobutane; perfluoromorpholine derivatives such as 4-trifluoromethylperfluoromorpholine for example sold under the name “PF 5052®” by the company 3M.

The expression “hydrocarbon oil” in the list of oils mentioned above is understood to mean any oil predominantly containing carbon and hydrogen atoms, and optionally ester, ether, fluorinated, carboxylic acid and/or alcohol groups.

A wax can be a lipophilic compound which is solid at room temperature (about 25° C.) and exhibits a reversible solid/liquid change of state and which has a melting point greater than about 40° C. and capable of going up to 200° C., and which exhibits, in the solid state, an anisotropic crystalline organization. The animal and plant waxes comprise, as essential constituents, esters of carboxylic acids and alcohols with long chains. In a general way, the size of the crystals of the wax is such that the crystals diffract and/or scatter light, conferring on the composition comprising them a cloudy appearance that is opaque to a greater or lesser degree. Upon heating the wax to its melting point, it is possible to make it miscible with oils and to form a microscopically homogeneous mixture, but upon bringing the temperature of the mixture to room temperature, recrystallization of the wax in the oils of the mixture is obtained, which is capable of being microscopically and macroscopically (opalescence) detected.

As waxes which can be used, there may be mentioned waxes of animal origin such as beeswax, spermaceti, lanolin wax and lanolin derivatives; plant waxes such as sunflower, rice and apple waxes, carnauba wax, candelilla wax, ouricury wax, Japan wax, cocoa butter or cork fibre or sugarcane waxes; mineral waxes, for example paraffin, petroleum jelly and lignite waxes, or microcrystalline waxes, ceresin or ozokerite; synthetic waxes such as polyethylene waxes, Fischer-Tropsch waxes; waxy fatty acid esters; waxy fatty alcohols such as for example myristyl, cetyl, stearyl, arachidyl, behenyl and erucyl alcohols, and mixtures thereof.

As vegetable oil, there may be mentioned sweet almond oil, avocado oil, castor oil, olive oil, jojoba liquid wax, sunflower oil, wheat germ oil, sesame oil, groundnut oil, grapeseed oil, soybean oil, rapeseed oil, safflower oil, copra oil, maize oil, hazelnut oil, palm oil, apricot kernel oil, calophyllum oil, evening primrose oil, shea butter, rice bran oil, wheat germ oil, passion flower oil and rye oil.

As animal oil, there may be mentioned, for example, perhydrosqualene.

As mineral oil, there may be mentioned, for example, paraffin oil and liquid petroleum jelly.

As synthetic oil, there may be mentioned, for example, squalane, poly(L-olefins) such as isododecane, isohexadecane, transesterified vegetable oils, fluorinated oils and fatty esters.

The expression fatty esters denotes the compounds of formula RₐCOOR_{b} in which Rₐ represents the residue of a saturated or unsaturated, hydroxylated or non-hydroxylated, linear or branched higher acid containing from 4 to 29 carbon atoms and R_{b} represents a saturated or unsaturated, linear or branched hydrocarbon chain containing from 3 to 30 carbon atoms, the total number of carbon atoms of the ester being greater than 10. By way of non-limiting examples, there may be mentioned, for example, purcellin oil (stearyl octanoate), isopropyl myristate, isopropyl palmitate, butyl stearate, hexyl laurate, isononyl isononanoate, 2-ethylhexyl palmitate, 2-hexyldecyl laurate, 2-octyldecyl palmitate, 2-octyldodecyle myristate, isostearyl neopentanoate or tridecyl neopentanoate.

The fluorinated oils may be partially hydrocarbon-based and/or silicone-based, such as for example those described in the document JP-A-2-295912.

The representative fatty alcohols comprise, inter alia, myristyl, cetyl, stearyl, arachidyl, behenyl and erucyl alcohols.

### Surfactants

According to at least one embodiment, the composition contains less than 5% by weight in total of anionic surfactants and non-ionic surfactants, such as less than 3% by weight, and further such as less than 1% by weight, even further such as less than 0.5% by weight relative to the total weight of the composition.

As a further example, the anionic surfactants and the non-ionic surfactants are completely absent from the compositions.

The composition may contain one or more anionic and/or non-ionic surfactants, as long as the total amount of anionic surfactants and of non-ionic surfactants, for example, remains less than the values indicated above.

By way of example of anionic surfactants which may be present in the compositions, there may be mentioned (non-limiting list) the salts (for example the alkali metal, such as sodium, salts, ammonium salts, amine salts, amino alcohol salts or alkaline-earth metal (such as magnesium) salts) of the following compounds: alkyl sulphates, alkyl ether sulphates, alkyl amidoether sulphates, alkyl aryl polyether sulphates, monoglyceride sulphates; alkyl sulphonates, alkyl phosphates, alkyl amide sulphonates, alkyl aryl sulphonates, α-olefin sulphonates, paraffin sulphonates; alkyl sulphosuccinates; alkyl ether sulphosuccinates, alkyl amide sulphosuccinates; alkyl sulphosuccinamates; alkyl sulphoacetates; alkyl ether phosphates, acyl sarcosinates; acyl isethionates and N-acyltaurates, the alkyl or acyl radical of all these various compounds containing from 12 to 20 carbon atoms, and the aryl radical denoting a phenyl or benzyl group.

Among the anionic surfactants, mention may also be made of the salts of fatty acids such as oleic, ricinoleic, palmitic and stearic acid salts; the acids of copra oil or hydrogenated copra oil; acyl lactylates whose acyl radical contains 8 to 20 carbon atoms.

Mention may also be made of weakly anionic surfactants, such as alkyl D-galactoside uronic acids and their salts and polyoxyalkylenated (C₆-C₂₄)alkyl ether carboxylic acids, polyoxyalkylenated (C₆-C₂₄)alkyl aryl ether carboxylic acids, polyoxyalkylenated (C₆-C₂₄)alkyl aminoether carboxylic acids and the salts of these acids, for example, those containing from 2 to 50 ethylene oxide groups and mixtures thereof.

The non-ionic surfactants which may be present in the compositions are compounds well known per se (see for example in this regard “Handbook of Surfactants” by M. R. PORTER, Blackie & Son publishers (Glasgow and London), 1991, pp 116-178). They are chosen, for example, from polyethoxylated, polypropoxylated or polyglycerolated alcohols and fatty alcohols, polyethoxylated, polypropoxylated or polyglycerolated alpha-diols, polyethoxylated, polypropoxylated or polyglycerolated (C₁₋₂₀)alkylphenols or polyethoxylated, polypropoxylated or polyglycerolated fatty acids, the fatty chain containing, for example, from 8 to 18 carbon atoms, it being possible for the number of ethylene oxide or propylene oxide groups to range from 2 to 50 and it being possible for the number of glycerol groups to range from 2 to 30.

Mention may also be made of the condensates of ethylene oxide and propylene oxide with fatty alcohols; polyethoxylated fatty amides having from 2 to 30 ethylene oxide units, polyglycerolated fatty amides containing on average from 1 to 5 glycerol groups such as from 1.5 to 4, ethoxylated fatty acid esters of sorbitan having from 2 to 30 ethylene oxide units, fatty acid esters of sucrose, fatty acid esters of polyethylene glycol, alkyl polyglycosides, polyethoxylated vegetable oils, N—(C₆₋₂₄ alkyl)glucamine derivatives, amine oxides such as (C₁₀₋₁₄ alkyl)amine oxides or N—(C₁₋₁₄ acyl)-aminopropylmorpholine oxides.

The composition may moreover contain one or more amphoteric or zwitterionic surfactants.

The amphoteric or zwitterionic surfactants which can be used in the compositions comprise, for example, secondary or tertiary aliphatic amine derivatives in which the aliphatic group is a linear or branched chain containing from 8 to 22 carbon atoms and containing at least one anionic group such as, for example, a carboxylate, sulphonate, sulphate, phosphate or phosphonate group. Mention may also be made of (C₈₋₂₀)alkyl betaines, suiphobetaines, (C₈₋₂₀ alkyl)amido(C₆₋₈ alkyl)betaines or (C₈₋₂₀ alkyl)amido(C₆₋₈ alkyl)sulphobetaines.

Among the amine derivatives, there may be mentioned the products marketed under the name MIRANOL®, as described in U.S. Pat. No. 2,528,378 and U.S. Pat. No. 2,781,354 and classified in the CTFA dictionary, 3rd edition, 1982, under the names Amphocarboxyglycinate and Amphocarboxypropionate having the respective structures (1) and (2):

Rₐ—CONHCH₂CH₂—N(R_{b})(R_{c})(CH₂COO⁻) (1)

in which:

Rₐ represents an alkyl group derived from an acid Rₐ—COOH present in hydrolysed copra oil, a heptyl, nonyl or undecyl group,

R_{b} represents a beta-hydroxyethyl group, and

R_{C} represents a carboxymethyl group;

and

Rₐ′—CONHCH₂CH₂—N(B)(B′) (2)

in which:

B represents —CH₂CH₂OX′,

B′ represents —(CH₂)_{z}—Y′, with z=1 or 2,

X′ represents the group —CH₂CH₂—COOH or a hydrogen atom,

Y′ represents —COOH or the group —CH₂—CHOH—SO₃H,

Rₐ′ represents an alkyl group of an acid Rₐ′—COOH present in copra oil or in hydrolysed linseed oil, an alkyl group, such as C₁₇ and its iso form, an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of example, there may be mentioned the cocoamphodiacetate marketed by the company RHODIA under the trade name MIRANOL® C2M concentrate.

The composition may comprise from 0.01 to 10% by weight of amphoteric or zwitterionic surfactant(s), such as from 0.05 to 4% by weight, relative to the total weight of the composition.

According to at least one embodiment, the composition can be a non-detergent composition, that is to say that it contains less than 5% by weight in total of detergent surfactants, such as less than 3% by weight, further such as less than 1% by weight, and even further such as less than 0.5% by weight, relative to the total weight of the composition. The expression detergent surfactants denotes anionic, non-ionic, amphoteric and zwitterionic surfactants.

The composition may moreover contain one or more cationic surfactants.

The cationic surfactants which may be used in the compositions comprise, for example, salts of primary, secondary or tertiary fatty amines, optionally polyoxyalkylenated, quaternary ammonium salts, and mixtures thereof.

As quaternary ammonium salts, there may be mentioned, for example:

those corresponding to the following general formula (IX):

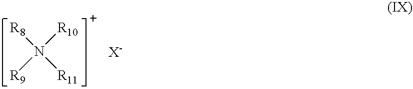

in which the radicals R₈ to R₁₁, which may identical or different, represent a linear or branched aliphatic radical containing from 1 to 30 carbon atoms, or an aromatic radical such as aryl or alkylaryl. The aliphatic radicals may contain heteroatoms such as in particular oxygen, nitrogen, sulphur and halogens. The aliphatic radicals are for example chosen from C₁₋₃₀ alkyl, C₁₋₃₀alkoxy, (C₂-C₆) polyoxyalkylene, C₁₋₃₀ alkylamide, (C₁₂-C₂₂alkyl)amido(C₂-C₆alkyl), (C₁₂-C₂₂)alkyl acetate, and C₁₋₃₀ hydroxyalkyl; X is an anion chosen from the group comprising halides, phosphates, acetates, lactates, (C₂-C₆)alkyl sulphates, alkyl- or alkylaryl sulphonates.

Among the quaternary ammonium salts of formula (I), exemplary mention may be made of, on the one hand, tetraalkylammonium chlorides such as, for example, dialkyldimethylammonium or alkyltrimethylammonium chlorides in which the alkyl radical contains about 12 to 22 carbon atoms, for example, behenyltrimethylammonium, distearyidimethylammonium, cetyltrimethylammonium and benzyldimethylstearylammonium chlorides or alternatively, on the other hand, palmitylamidopropyltrimethylammonium chloride or stearamidopropyldimethyl(myristyl acetate)ammonium chloride marketed under the name CERAPHYL® 70 by the company VAN DYK.

quaternary ammonium salts of imidazoline, such as for example those of the following formula (X):

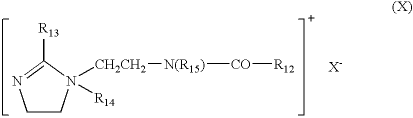

in which R₁₂ represents an alkenyl or alkyl radical containing from 8 to 30 carbon atoms, for example derived from tallow fatty acids, R₁₃ represents a hydrogen atom, a C₁-C₄ alkyl radical or an alkenyl or alkyl radical containing from 8 to 30 carbon atoms, R₁₄ represents a C₁-C₄ alkyl radical, R₁₅ represents a hydrogen atom, a C₁-C₄ alkyl radical, X⁻ is an anion chosen from the group comprising halides, phosphates, acetates, lactates, alkyl sulphates, alkyl or alkylaryl sulphonates. Preferably, R₁₂ and R₁₃ denote a mixture of alkenyl or alkyl radicals containing from 12 to 21 carbon atoms, for example derived from tallow fatty acids, R₁₄ denotes a methyl radical, R₁₅ denotes a hydrogen atom. Such a product is for example marketed under the name REWOQUAT® W 75 by the company REWO;

quaternary diammonium salts of the following formula (XI):

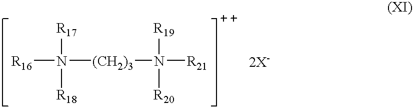

in which R₁₆ denotes an aliphatic radical containing about 16 to 30 carbon atoms; R₁₇, R₁₈, R₁₉, R₂₀ and R₂₁, which are identical or different, are chosen from a hydrogen atom and an alkyl radical containing from 1 to 4 carbon atoms; and X is an anion chosen from the group comprising halides, acetates, phosphates, nitrates and methyl sulphates. Such quaternary diammonium salts, for example, comprise propane tallowediammonium dichloride;

quaternary ammonium salts containing at least one ester functional group, such as those of the following formula (XII):

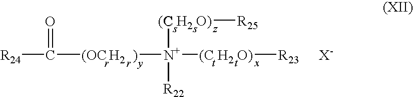

in which:

R₂₂ is chosen from C₁-C₆ alkyl radicals and C₁-C₆ hydroxyalkyl or dihydroxyalkyl radicals;

R₂₃ is chosen from:

the radical

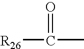

the saturated or unsaturated, linear or branched C₁-C₂₂ hydrocarbon radicals R₂₇,

the hydrogen atom,

R₂₅ is chosen from:

the radical

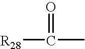

the saturated or unsaturated, linear or branched, C₁-C₆ hydrocarbon radicals R₂₉,

the hydrogen atom,

R₂₄, R₂₆ and R₂₈, which are identical or different, are chosen from saturated or unsaturated, linear or branched, C₇-C₂₁ hydrocarbon radicals;

r, s and t, which are identical or different, are integers equal to 2 to 6;

y is an integer equal to 1 to 10;

x and z, which are identical or different, are integers equal to 0 to 10;

X⁻ is a simple or complex, organic or inorganic anion;

provided that the sum x+y+z equals 1 to 15, that when x is equal to 0, then R₂₃ denotes R₂₇ and that when z is equal to 0, then R₂₅ denotes R₂₉.

The alkyl radicals R₂₂ may be linear or branched, and for example, linear.

For example, R₂₂ denotes a methyl, ethyl, hydroxyethyl or dihydroxypropyl radical, and more particularly a methyl or ethyl radical.

For example, the sum x+y+z equals 1 to 10.

When R₂₃ is a hydrocarbon radical R₂₇, it may be long and have from 12 to 22 carbon atoms, or short and have from 1 to 3 carbon atoms.

When R₂₅ is a hydrocarbon radical R₂₉, it may have 1 to 3 carbon atoms.

For example, R₂₄, R₂₆ and R₂₈, which are identical or different, are chosen from saturated and unsaturated, linear and branched C₁₁-C₂₁ hydrocarbon radicals, such as from saturated and unsaturated, linear and branched C₁₁-C₂₁ alkyl and alkenyl radicals.

For example, x and z, which are identical or different, are equal to 0 or 1.

For example, y is equal to 1.

For example, r, s and t, which are identical or different, are equal to 2 or 3, such as are equal to 2.

The anion may be a halide (chloride, bromide or iodide) or an alkyl sulphate, such as methyl sulphate. It is possible, however, to use methanesulphonate, phosphate, nitrate, tosylate, an organic acid-derived anion such as acetate or lactate or any other anion compatible with ammonium having an ester functional group.

The anion X⁻ is, for example, chloride or methyl sulphate.

Use is made, for example, in the composition of the ammonium salts of formula (XII) in which:

R₂₂ denotes a methyl or ethyl radical,

x and y are equal to 1;

z is equal to 0 or 1;

r, s and t are equal to 2;

R₂₃ is chosen from:

the radical

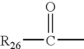

methyl, ethyl or C₁₄-C₂₂ hydrocarbon radicals, and

the hydrogen atom;

R₂₅ is chosen from:

the radical

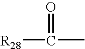

the hydrogen atom;

R₂₄, R₂₆ and R₂₈, which are identical or different, are chosen from saturated and unsaturated, linear and branched C₁₃-C₁₇ hydrocarbon radicals, such as from saturated and unsaturated, linear and branched C₁₃-C₁₇ alkyl and alkenyl radicals.

For example, the hydrocarbon radicals are linear.

There may be mentioned for example the compounds of formula (XII) such as the salts (for example, chloride or methyl sulphate) of diacyloxyethyldimethylammonium, diacyloxyethylhydroxyethyl-methylammonium, monoacyloxyethyldihydroxyethylmethylammonium, triacyloxyethylmethylammonium, monoacyloxyethylhydroxyethyl-dimethylammonium and mixtures thereof. The acyl radicals may have 14 to 18 carbon atoms and may be derived from a vegetable oil such as palm or sunflower oil. When the compound contains several acyl radicals, the latter may be identical or different.

These products are obtained, for example, by direct esterification of triethanolamine, triisopropanolamine, alkyldiethanolamine or alkyldiisopropanolamine optionally oxyalkylenated on fatty acids or on mixtures of fatty acids of plant or animal origin, or by transesterification of their methyl esters. This esterification is followed by quaternization with the aid of an alkylating agent such as an alkyl (preferably methyl or ethyl) halide, a dialkyl (preferably methyl or ethyl) sulphate, methyl methanesulphonate, methyl para-toluenesulphonate, glycol or glycerol chlorohydrin.

Such compounds are for example marketed under the names DEHYQUART® by the company HENKEL, STEPANQUAT® by the company STEPAN, NOXAMIUM® by the company CECA, REWOQUAT® WE 18 by the company REWO-WITCO.

The composition may contain, for example, a mixture of quaternary ammonium mono-, di- and triester salts with a majority of diester salts by weight.

As mixture of ammonium salts, use may be made for example of the mixture containing 15 to 30% by weight of acyloxyethyl-dihydroxyethylmethylammonium methyl sulphate, 45 to 60% of diacyloxyethylhydroxyethylmethylammonium methyl sulphate and 15 to 30% of triacyloxyethylmethylammonium methyl sulphate, the acyl radicals having from 14 to 18 carbon atoms and being derived from optionally partially hydrogenated palm oil.

Use may also be made of the ammonium salts containing at least one ester functional group, which are described in U.S. Pat. No. 4,874,554 and U.S. Pat. No. 4,137,180.

The composition may comprise from 0.01 to 10% by weight of cationic surfactant(s), such as from 0.05 to 4% by weight, relative to the total weight of the composition.

The compositions may also additionally comprise one or more silicones in soluble, dispersed or microdispersed form. The silicones may then present in a quantity ranging from 0.01 to 10% by weight, and such as from 0.1 to 5% by weight, relative to the total weight of the composition.

By way of example there may be mentioned silicone oils, such as for example linear or cyclic polydimethylsiloxanes.

The compositions may be packaged for example in a pot, in a tube, in a pump dispenser, or in an aerosol device customarily used in the cosmetic field.

The compositions may, when they are intended to be packaged in an aerosol-type device, contain one or more propellant gases.

The propellant gas may then be chosen, for example, from dimethyl ether, C₃ to C₅ alkanes, halogenated hydrocarbons, and mixtures thereof.

The compositions may additionally contain one or more additives chosen from pearlescent agents; opacifying agents; plasticizers; sunscreens; perfumes; colorants; preservatives; pH-stabilizing agents; acids; bases; polyols (for example glycols); inorganic fillers; glitter, and any other additive conventionally used in the cosmetic field.

Persons skilled in the art will be careful to choose the optional additives and their quantities such that they do not impair the properties of the compositions of the present invention.

These additives may be present in the composition in a quantity ranging from 0 to 50% by weight relative to the total weight of the composition.

The compositions may be provided, inter alia, in the form of liquids which are thickened to a greater or lesser degree, gels, creams, pastes or mousses.

For example, they are provided in the form of gels.

The composition may also be provided in the form of a composition in two parts, intended to be mixed at the time of use.

Another embodiment of the present invention is therefore also a two-part cosmetic composition, comprising,

a first part comprising, in a cosmetically acceptable medium, one or more vinylformamide/vinylformamine copolymers as described above, which comprises:

from 10 to less than 95 mol % of units of the following formula A:

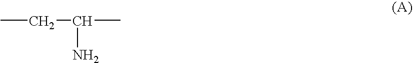

and from 90 to greater than 5 mol % of units of the following formula B:

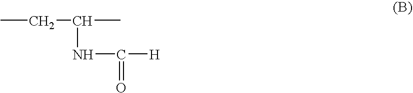

and

a second part comprising, in a cosmetically acceptable medium, one or more thickening polymers different from the vinylformamide/vinylformamine copolymers.

For example, the composition resulting from the mixing of the said first and second parts comprises less than 5% by weight in total of anionic surfactants and non-ionic surfactants.

The description made above of the various ingredients of the one-part composition also applies to the two-part composition, it being possible for the said ingredients to be present in either part with the exception of the vinylformamide/vinylformamine copolymer(s) and of the thickening polymer(s), which are packaged separately. Likewise, the above description of the amounts and ratios by weight of the various ingredients, including the vinylformamide/vinylformamine copolymer(s) and the thickening polymer(s) also applies to the two-part composition, it being understood that these amounts and ratios by weight apply to the final composition obtained after mixing the two parts.

Moreover, the two-part composition may be packaged in a multicompartment device or “kit”.

The present disclosure therefore also relates to a device comprising at least two compartments, wherein:

a first compartment comprises at least one first composition comprising, in a cosmetically acceptable medium, one or more vinylformamide/vinylformamine copolymers which comprises:

from 10 to 95 mol % of units of the following formula A:

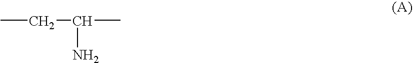

and from 90 to 5 mol % of units of the following formula B:

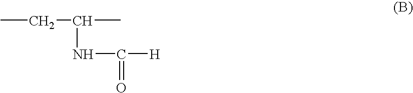

and

a second compartment comprises at least one second composition comprising, in a cosmetically acceptable medium, one or more thickeners different from the vinylformamide/vinylformamine copolymers.

The composition may be used for the cosmetic treatment of the hair. For example, it may be used for hair styling, for example, for shaping and/or fixing the hair style.

According to at least one embodiment, it is used for hair styling and the simultaneous conditioning of the hair.

The present disclosure also relates to a method for the cosmetic treatment of the hair, for example a method for hair care, or a method for shaping and/or maintaining the hair style, which comprises

applying to the hair an effective quantity of a composition comprising, in a cosmetically acceptable aqueous medium:

one or more vinylformamide/vinylformamine copolymers comprising:

from 10 to 95 mol % of units of the following formula A:

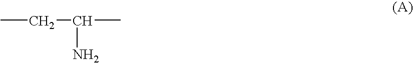

and from 90 to 5 mol % of units of the following formula B:

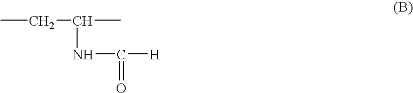

and

one or more thickening polymers different from the vinylformamide/vinylformamine copolymers.

then in performing an optional rinse after an optional leave-in time.

For example, the composition is not rinsed off.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The following examples are given by way of illustration of the present invention. In these examples, all the quantities are indicated in percent by weight of active material (AM) relative to the total weight of the composition.

### EXAMPLES

These examples illustrate the formulation of hair styling gels in accordance with the invention.

A first gel was prepared from the ingredients indicated in the table below:

| | |
|---|---|
| 30% hydrolysed polyvinylformamide (1) | 5% |
| Guar gum (2) | 2% |
| PRESERVATIVE | 0.3% |
| WATER | qs 100% |

A second gel was prepared from the ingredients indicated in the table below:

| | |
|---|---|
| 30% hydrolysed polyvinylformamide (1) | 6% |
| ACRYLIC ACID/VINYLPYRROLIDONE/LAURYL | 2% |
| METHACRYLATE TERPOLYMER ACRYLIDONE | |
| LM (ISP) | |
| Triethanolamine | qs pH = 8 |
| WATER | qs 100% |

(1) marketed under the name LUPAMIN 9030 by the company BASF.

(2) marketed under the name JAGUAR HP 105 by the company RHODIA.

### Results Obtained:

The performances of the two compositions described above were evaluated by professionals, on panels of models.

These compositions made it possible to obtain very good fixing of the hair, with excellent retention over time and good resistance to mechanical stresses.

In addition, these compositions were found to give the hair excellent cosmetic properties, in particular in terms of softness.

The following two compositions were prepared:

| | | Composition 2 (not in |
|---|---|---|
| | Composition 1 | accordance with the |
| | (invention) | invention) |
| 30% hydrolysed | 5% am | — |
| polyvinylformamide (1) | | |
| Poly-N-vinylformamide (2) | — | 5% am |
| Guar gum (3) | 2% am | 2% am |
| Demineralized water | qs 100 | qs 100 |

(1) marketed under the name LUPAMIN 9030 by the company BASF

(2) marketed under the name LUPAMIN 9000 by the company BASF

(3) marketed under the name JAGUAR HP 105 by the company RHODIA

The preceding two compositions were applied, at the rate of 1 g of formulation per lock, to 2.7 g locks of wet chestnut brown hair shampooed, rinsed and wrung beforehand.

Finally, the non-rinsed locks were then dried under a hood dryer for 30 minutes, and then disentangled.

A panel of 7 testers evaluated the smooth character of the hair treated with compositions 1 or 2 by classing them according to this criterion: rank 1 for the lock with the hair having the smoothest feel, rank 2 for the lock with the hair having the least smooth feel.

The 7 evaluators found a smoother feel with the composition 1 according to the invention.

Rank sum: 7 for composition 1, 14 for composition 2

According to the KRAMER test (A non parametric ranking method for the statistical evaluation of sensory data, chemical Senses and Flavor (1974) 121-123), at the 5% threshold, 7 being less than the interval 8-13, the feel is significantly smoother with the composition 1 according to the invention.

What is claimed is:

1. Cosmetic composition comprising, in a cosmetically acceptable medium:
one or more vinylformamide/vinylformamine copolymers comprising:
from 10 to less than 95 mol % of units of the following formula A: 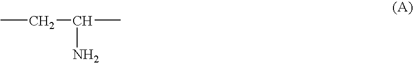
and from 90 to greater than 5 mol % of units of the following formula B: 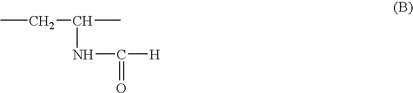
and
one or more thickening polymers different from the vinylformamide/vinylformamine copolymers.

2. Composition according to claim 1, characterized in that the one or more vinylformamide/vinylformamine copolymers comprise from 10 to 60 mol % of unit of formula A.

3. Composition according to claim 2, characterized in that the one or more vinylformamide/vinylformamine copolymers comprise from 20 to 40 mol % of unit of formula A.

4. Composition according to claim 1, characterized in that the one or more vinylformamide/vinylformamine copolymers comprise one or more additional monomer units, the latter representing less than 20 mol % of the copolymer.

5. Composition according to any one of claims 1, characterized in that the one or more vinylformamide/vinylformamine copolymers consist solely of units of formula A and of units of formula B.

6. Composition according to claim 1, characterized in that the one or more vinylformamide/vinylformamine copolymers are present in amounts ranging from 0.1 to 25% by weight, relative to the total weight of the composition.

7. Composition according to claim 6, characterized in that the one or more vinylformamide/vinylformamine copolymers are present in amounts ranging from 0.5 to 20% by weight, relative to the total weight of the composition.

8. Composition according to claim 1, characterized in that the one or more thickening polymers are non-associative.

9. Composition according to claim 1, characterized in that the one or more thickening polymers are associative.

10. Composition according to claim 8, characterized in that the one or more thickening polymers are chosen from
crosslinked homopolymers and copolymers of acrylic and methacrylic acid,
crosslinked homopolymers of 2-acrylamido-2-methylpropanesulphonic acid and their crosslinked copolymers of acrylamide,
homopolymers of ammonium acrylate and copolymers of ammonium acrylate and acrylamide,
non-ionic guar gums,
biopolysaccharide gums of microbial origin,
gums derived from plant exudates,
celluloses, and
pectins and alginates.

11. Composition according to claim 10, characterized in that the celluloses are chosen from hydroxypropyl- and carboxymethylcelluloses.

12. Composition according to claim 9, characterized in that the one or more associative thickening polymers is an anionic copolymer of an α,β-monoethylenically unsaturated carboxylic acid and an ester of an α,β-monoethylenically unsaturated carboxylic acid and an oxyalkylenated fatty alcohol.

13. Composition according to claim 9, characterized in that the one or more associative thickening polymers is a copolymer of an α,β-monoethylenically unsaturated carboxylic acid and an ester of a fatty alcohol and an α,β-monoethylenically unsaturated carboxylic acid and vinylpyrrolidone.

14. Composition according to claim 9, characterized in that the one or more associative thickening polymer is non-ionic, chosen from:
(1) celluloses modified with groups containing at least one fatty chain;
(2) hydroxypropyl guars modified with groups containing at least one fatty chain;
(3) copolymers of vinylpyrrolidone and hydrophobic monomers having a fatty chain;
(4) copolymers of C₁-C₆ alkyl methacrylates or acrylates and amphiphilic monomers containing at least one fatty chain;
(5) copolymers of hydrophilic methacrylates or acrylates and hydrophobic monomers containing at least one fatty chain;
(6) polyether-polyurethanes containing in their chain both polyoxyethylenated hydrophilic blocks and hydrophobic blocks; and
(7) polymers having an aminoplast ether backbone possessing at least one fatty chain.

15. Composition according to claim 14, characterized in that the polyether-polyurethanes are chosen from polyether-polyurethanes containing at least two lipophilic hydrocarbon chains having from 6 to 30 carbon atoms, separated by a hydrophilic block, it being possible for the hydrocarbon chains to be pendant chains or chains at the end of said hydrophilic block.

16. Composition according to claim 9, characterized in that the one or more thickening polymers are chosen from cationic associative polymers chosen from
quaternized celluloses modified by groups containing at least one fatty chain,
quaternized hydroxyethylcelluloses modified by groups containing at least one fatty chain and
polyurethanes.

17. Composition according to claim 1, characterized in that the one or more thickeners are present in the compositions in amounts ranging from 0.01 to 20% by weight, relative to the total weight of the composition.

18. Composition according to claim 17, characterized in that the one or more thickeners are present in the compositions in amounts ranging from 0.05 to 5% by weight, relative to the total weight of the composition.

19. Composition according to claim 17, characterized in that the one or more thickeners are present in the compositions in amounts ranging from 1 to 4% by weight, relative to the total weight of the composition.

20. Composition according to claim 1, characterized in that the weight ratio of the one or more vinylformamide/vinylformamine copolymer(s), on the one hand, to the one or more thickening polymers, on the other hand, ranges from 0.1 to 20

21. Composition according to claim 20, characterized in that the weight ratio ranges from 1 to 15.

22. Composition according to claim 20, characterized in that the weight ratio ranges from 1 to 8.

23. Composition according to claim 1, characterized in that the cosmetically acceptable medium comprises water and/or one or more cosmetically acceptable solvents chosen from C₁-C₄ lower alcohols, polyols, polyol ethers, C₅-C₁₀ alkanes, C₃₋₄ ketones, C₁-C₄ alkyl acetates dimethoxyethane, and diethoxyethane.

24. Composition according to claim 1, characterized in that the composition further comprises from 0.01 to 10% by weight of one or more amphoteric surfactant(s), relative to the total weight of the composition.

25. Composition according to claim 1, characterized in that the composition further comprises from 0.01 to 10% by weight of one or more zwitterionic surfactant(s), relative to the total weight of the composition.

26. Composition according to claim 1, characterized in that the composition further comprises from 0.01 to 10% by weight of one or more cationic surfactant(s), relative to the total weight of the composition.

27. Composition according to claim 1, characterized in that the composition additionally comprises one or more propellants.

28. Composition according to claim 1, characterized in that the composition additionally contains one or more additives chosen from pearlescent agents; opacifying agents; plasticizers; sunscreens; perfumes; colorants; preservatives; pH-stabilizing agents; acids; bases; polyols; inorganic fillers; glitter, and any other additive conventionally used in the cosmetic field.

29. Composition according to claim 1, characterized in that the composition is provided in the form of a thickened liquid which is a gel, a cream, a paste or a mousse.

30. Composition according to claim 29, characterized in that the composition is provided in the form of a gel.

31. Composition according to claim 1, characterized in that the composition comprises less than 5% by weight in total of anionic surfactants and non-ionic surfactants.

32. Two-part cosmetic composition, comprising:
a first part comprising, in a cosmetically acceptable medium, one or more vinylformamide/vinylformamine copolymers, which comprises:
from 10 to less than 95 mol % of units of the following formula A: 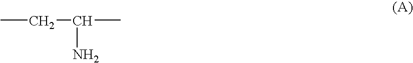
and from 90 to greater than 5 mol % of units of the following formula B: 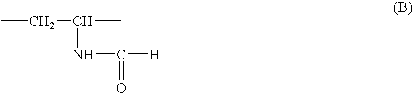
and
a second part comprising, in a cosmetically acceptable medium, one or more thickeners different from the vinylformamide/vinylformamine copolymers.

33. Composition according to claim 32, characterized in that the said first and second parts combined comprise less than 5% by weight in total of anionic surfactants and non-ionic surfactants.

34. Device comprising at least two compartments, wherein:
a first compartment comprises at least one first composition comprising, in a cosmetically acceptable medium, one or more vinylformamide/vinylformamine copolymers which comprises:
from 10 to 95 mol % of units of the following formula A: 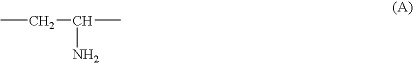
and from 90 to 5 mol % of units of the following formula B: 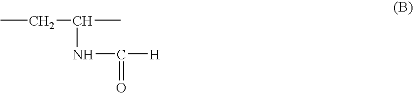
, and
a second compartment comprises at least one second composition comprising, in a cosmetically acceptable medium, one or more thickeners different from the vinylformamide/vinylformamine copolymers.

35. Method for the cosmetic treatment of the hair comprising
applying to the hair an effective quantity of a composition comprising, in a cosmetically acceptable aqueous medium:
one or more vinylformamide/vinylformamine copolymers comprising:
from 10 to 95 mol % of units of the following formula A: 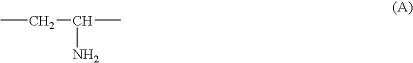
and from 90 to 5 mol % of units of the following formula B: 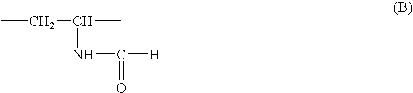
and
one or more thickening polymers different from the vinylformamide/vinylformamine copolymers.
and then performing an optional rinse after an optional leave-in time.

36. Method according to claim 35, characterized in that the composition is not rinsed off.

37. A method for shaping and/or fixing of the hairstyle, comprising applying to hair a cosmetic composition comprising, in a cosmetically acceptable aqueous medium:
one or more vinylformamide/vinylformamine copolymers comprising:
from 10 to 95 mol % of units of the following formula A: 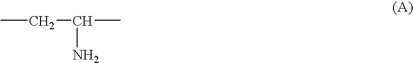
and from 90 to 5 mol % of units of the following formula B: 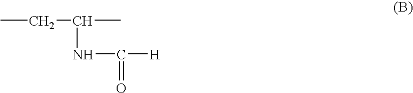
and
one or more thickening polymers different from the vinylformamide/vinylformamine copolymers.

38. A method for simultaneously styling and conditioning the hair comprising applying to the hair a cosmetic composition comprising, in a cosmetically acceptable aqueous medium:
one or more vinylformamide/vinylformamine copolymers comprising:
from 10 to 95 mol % of units of the following formula A: 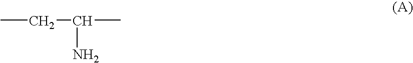
and from 90 to 5 mol % of units of the following formula B: 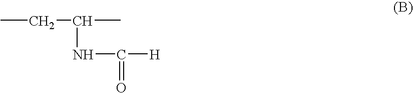
and
one or more thickening polymers different from the vinylformamide/vinylformamine copolymers.
